(19) United States
(12) Patent Application Publication
(10) Pub. No.: US 2019/0293621 A1
(43) Pub. Date: Sep. 26, 2019

(54) SENSOR SYSTEM INCLUDING AN INTEGRATED TEMPERATURE ADJUSTMENT ELEMENT

(71) Applicants: Michael Salerno, Edmond, OK (US); Jianmin Zhang, Edmond, OK (US); Sam Stroder, Edmond, OK (US); Qiliang Wang, Edmond, OK (US)

(72) Inventors: Michael Salerno, Edmond, OK (US); Jianmin Zhang, Edmond, OK (US); Sam Stroder, Edmond, OK (US); Qiliang Wang, Edmond, OK (US)

(73) Assignee: Baker Hughes, a GE company, LLC, Houston, TX (US)

(21) Appl. No.: 15/935,885

(22) Filed: Mar. 26, 2018

(51) Int. Cl. G01N 33/28; G01N 17/00

(52) U.S. Cl. CPC G01N 33/2823; G01N 17/008

(57) ABSTRACT

A sensor system including a body having an inlet, an outlet, and a fluid passage extending between the inlet and the outlet. A sensor is arranged at the fluid passage spaced from the outlet, and a temperature adjustment element is arranged in thermal communication with at least one of a portion of the fluid passage and the sensor.

### BACKGROUND

In the fluid handling industry, various sensors are employed to monitor fluid parameters at various process stages. In certain instances, monitoring fluid at an area of interest may present challenges. For example, locating a sensor on a tubular string at a fluid reservoir may be challenging. The sensor may be exposed to a harsh environment and, over time, may cease to function. Removing the tubular string to replace a sensor is a costly, time consuming effort. In addition to the cost of removing and re-inserting the tubular string, interruptions in production are costly.

Other challenges may be presented when mounting sensors to fluid treatment systems such as, for example, heat exchangers. Again, fluid treatment systems may present a harsh environment including high temperatures, harsh chemicals and the like that make accessing sensors difficult. It may also be impractical to install the sensor directly at the surface or system of interest. As such, maintenance and/or replacement of the sensors represents a costly undertaking. Accordingly, the art would be receptive to sensors that are capable of simulating an environment of a fluid or material of interest without actually being present in that environment.

### SUMMARY

Disclosed is a sensor system including a body having an inlet, an outlet, and a fluid passage extending between the inlet and the outlet. A sensor is arranged at the fluid passage spaced from the outlet, and a temperature adjustment element is arranged in thermal communication with at least one of a portion of the fluid passage and the sensor.

Also disclosed is a system including a fluid source and a passage extending from the fluid source. The passage includes a zone of interest. A sensor system is mounted to the passage remote from the zone of interest. The sensor system includes a body having an inlet, an outlet, and a fluid passage extending between the inlet and the outlet. A sensor is arranged at the fluid passage spaced from the outlet, and a temperature adjustment element is arranged in thermal communication with at least one of a portion of the fluid passage and the sensor

### BRIEF DESCRIPTION OF THE DRAWINGS

The following descriptions should not be considered limiting in any way. With reference to the accompanying drawings, like elements are numbered alike:

FIG. 1 depicts a resource exploration and recovery system including a sensor system, in accordance with an aspect of an exemplary embodiment;

FIG. 2 depicts a sensor system, in accordance with an aspect of an exemplary embodiment; and

FIG. 3 depicts a fluid treatment system including a sensor system, in accordance with another aspect of an exemplary embodiment.

### DETAILED DESCRIPTION

A detailed description of one or more embodiments of the disclosed apparatus and method are presented herein by way of exemplification and not limitation with reference to the Figures.

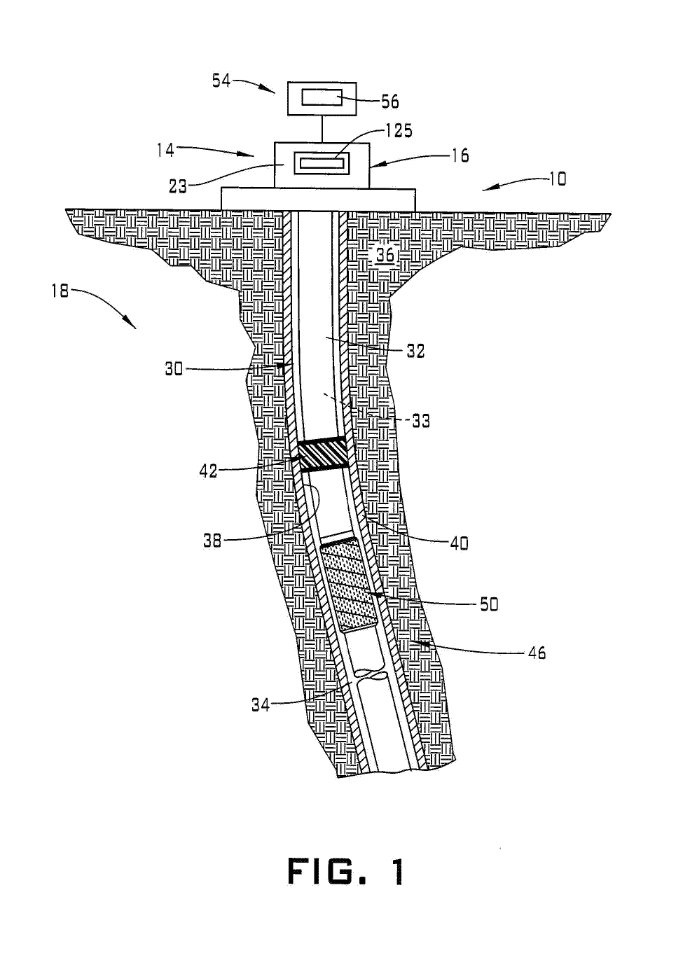

A resource exploration and recovery system, in accordance with an exemplary embodiment, is indicated generally at 10, in FIG. 1. Resource exploration and recovery system 10 should be understood to include well drilling operations, resource extraction and recovery, CO₂ sequestration, and the like. Resource exploration and recovery system 10 may include a first system 14 which, in some environments, may take the form of a surface system 16 operatively and fluidically connected to a second system 18 which, in some environments, may take the form of a downhole system. First system 14 may include a control system 23 that may provide power to, monitor, communicate with, and/or activate one or more downhole operations as will be discussed herein. Surface system 16 may include additional systems such as pumps, fluid storage systems, instrumentation and the like (not shown).

Second system 18 may include a tubular string 30, formed from one or more tubulars 32 defining a passage 33 that extends into a wellbore 34 formed in formation 36. Wellbore 34 includes an annular wall 38 which may be defined by a surface of formation 36, or a casing tubular 40 such as shown. Tubular string 32 may support a plurality of packer assemblies, one of which is indicated at 42 that separate wellbore 34 into a plurality of zones of interest 46 such as production and/or treatment zones. One or more screen assemblies 50 may be arranged in each production and/or treatment zone 46. In an embodiment, a fluid monitoring system 54 may be connected with the control system 23. Fluid monitoring system 54 may include a sensor system 56 that simulates environmental conditions, such as temperature, at the zone of interest while being located outside of formation 36. While shown as part of surface system 16, it should be understood that sensor system 56 may also be arranged downhole.

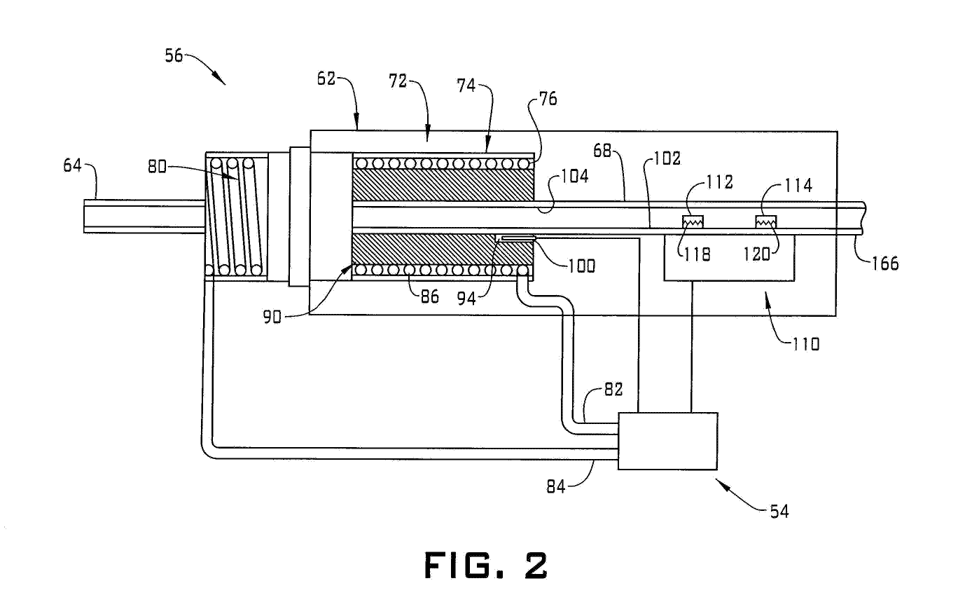

Referring to FIG. 2 and with continued reference to FIG. 1, sensor system 56 includes a body 62 having an inlet 64 and an outlet 66 fluidically connected through a fluid passage 68. Inlet 64 may be connected to a fluid source, such as tubular string 30. A heating system 72 may be arranged in body 62 and in thermal contact with fluid passage 68. Temperature adjustment system 72 may include a shell 74 having an interior 76 that houses a temperature adjustment element 80. Temperature adjustment element 80 include a first end 82, a second end 84 and an intermediate portion having a plurality of coils 86 extending therebetween. First and second ends 82 and 84 may be electrically connected to fluid monitoring system 54. Temperature adjustment element 80 may take on various forms including heating mechanisms, such as electrical resistive elements, microwaves, steam, induction and the like; or cooling systems that may employ a stream of gas such as air or a refrigerant.

In an embodiment, plurality of coils 86 may extend about an inner ring 90 which, in turn; extends about fluid passage 68. Inner ring 90 is formed from a thermally conductive material and may include a recess 94 within which may be positioned a thermocouple 100. Thermocouple 100 may be connected to control system 23 via fluid monitoring system 54 for monitoring, for example, a temperature of fluid passage 68. In an embodiment, fluid passage 68 includes an inner surface 102 that may be provided with a coating 104 resistant to a material of concern For example, coating 104 may represent an anti-scaling coating resistant to adhesion of inorganic scale, asphaltene, or paraffin that may precipitate from a fluid passing through fluid passage 68.

In accordance with an embodiment, sensor system 56 includes a sensor 110 that may include a first sensor element 112 and a second sensor element 114 that extends into fluid passage 68. Sensor 110 may be connected to fluid monitoring system 54 and configured to detect the material of concern. For example, sensor 110 may be configured to detect a presence of inorganic scale, asphaltene, or paraffin that has precipitated from a fluid passing through fluid passage 68. Sensor 110 may also be configured to detect a material of concern that has a reduced precipitation tendency at specific temperatures or in specific temperature ranges.

Temperature adjustment system 72 is configured to replicate environmental conditions at zone of interest 46. In this manner, sensor system 56 may be arranged remotely from zone of interest 46 yet still be configured to monitor fluids experiencing conditions similar to fluids at zone of interest 46. Further, as an alternative, or in addition to temperature adjustment element 80, each sensor element 112 and 114 may be temperature adjustable. For example, a first temperature adjustment member 118 may be incorporated into first sensor element 112 and a second temperature adjustment member 120 may be incorporated into second sensor element 114. It should be understood that as an alternative to integrated temperature adjustment members, other forms of temperature control may be employed for each sensor element 112 and 114.

In accordance with an exemplary embodiment, fluid monitoring system 54 may be connected to a feedback system 125 that may form part of control system 23. Feedback system 125 may receive inputs from sensor system 56 and control an introduction of chemicals that substantially reduce the formation of the material of concern. For example, feedback system 125 may control the introduction of anti-scale chemicals into system of tubulars 30.

At this point it should be understood that the exemplary embodiments describe a sensor system that may replicate conditions at a zone of interest yet be placed remotely from said zone. In this manner, fluid parameters at the zone of interest may be simulated without necessitating the placement of the sensor system in an area that is difficult and/or costly to access. The sensor system may be employed to control a feedback system that introduces chemicals to adjust parameters of the fluid passing from and/or through the zone of interest in order to reduce the material of concern and thereby substantially eliminate, for example, fouling.

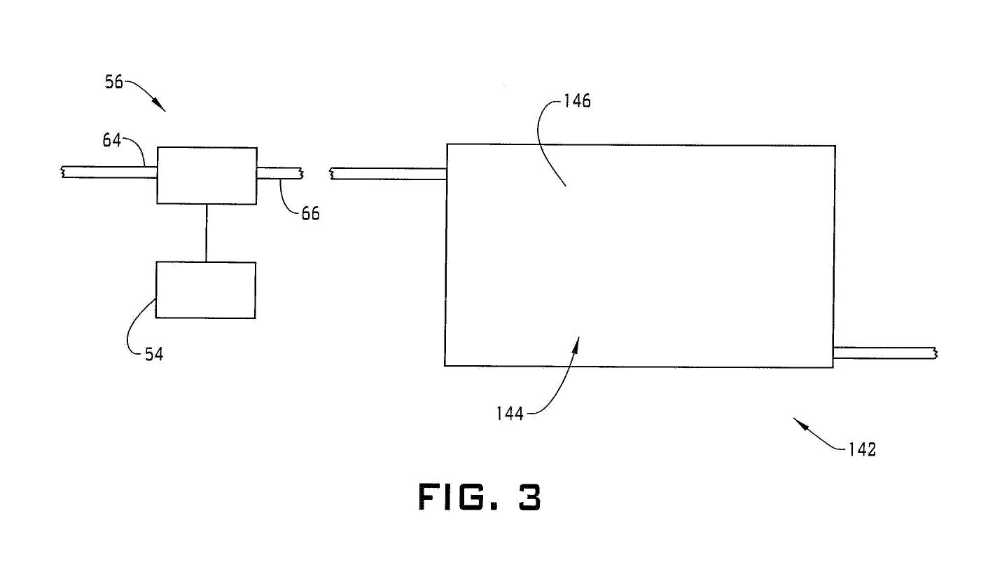

It should also be understood that while shown and described as a downhole system, exemplary embodiments may also include a fluid treatment system 142 such as shown in FIG. 3, wherein like reference numbers represent corresponding parts in the respective views. Fluid treatment system 142 may take the form of a heat exchanger 144 connected to a fluid source (not shown). Heat exchanger 144 includes a zone of interest 146, such as a heat exchange zone. Sensor system 56 may be positioned to remotely monitor fluid passing into fluid treatment system 142. Feedback system 125 may control an introduction of chemicals to counteract or substantially reduce the formation of a material of concern in fluid passing through fluid treatment system 142 in order to prolong an overall operational life and substantially reduce maintenance costs.

By simulating the conditions at a zone of interest via physically altering, for example, temperature conditions of the fluid and/or the sensor itself, more accurate and meaningful measurements can be obtained, without relying on theoretical correlations or predetermined transfer functions. In addition, the localized heating of the environment around the sensor may tend to promote inorganic scale formation, while also decreasing deposition of hydrocarbon-based foulants. In this way, the sensitivity of the sensor can be improved for the material of interest. In this way, one embodiment of the invention can be the use of multiple sensors at various temperatures to deconvolute fouling tendency into various constituents. Further, while described as a single sensor, it should be understood that multiple sensors may be employed to determine the material or materials of concern.

Set forth below are some embodiments of the foregoing disclosure:

### Embodiment 1

A sensor system including a body having an inlet, an outlet, and a fluid passage extending between the inlet and the outlet. A sensor is arranged at the fluid passage spaced from the outlet, and a temperature adjustment element arranged in thermal communication with at least one of a portion of the fluid passage and the sensor.

### Embodiment 2

The sensor system according any prior embodiment, wherein the temperature adjustment element includes a plurality of coils extending about the portion of the fluid passage.

### Embodiment 3

The sensor system according to any prior embodiment, further including a thermocouple arranged in the body for monitoring a temperature of the fluid passage.

### Embodiment 4

The sensor system according to any prior embodiment, further comprising: an inner ring arranged about a portion of the fluid passage, the temperature adjustment element extending about at least a portion of the inner ring.

### Embodiment 5

The sensor system according to any prior embodiment, wherein the thermocouple is arranged in the inner ring. The terms “about” and “substantially” are intended to include the degree of error associated with measurement of the particular quantity based upon the equipment available at the time of filing the application. For example, “about” and/or “substantially” can include a range of ±8% or 5%, or 2% of a given value.

### Embodiment 6

The sensor system according to any prior embodiment, wherein the sensor includes at least one sensor element extending into the fluid passage.

### Embodiment 7

The sensor system according to any prior embodiment, wherein the fluid passage includes an inner surface extending from the inlet to the outlet, the inner surface being provided with a coating resistant to a material of concern.

### Embodiment 8

The sensor system according to any prior embodiment, wherein the coating comprises an anti-scaling coating.

### Embodiment 9

A system including a fluid source, and a passage extending from the fluid source. The passage includes a zone of interest, and a sensor system mounted to the passage remote from the zone of interest. The sensor system includes a body including an inlet, an outlet, and a fluid passage extending between the inlet and the outlet, a sensor arranged at the fluid passage spaced from the outlet, and a temperature adjustment element arranged in thermal communication with at least one of a portion of the fluid passage and the sensor.

### Embodiment 10

The system according to any prior embodiment, wherein the temperature adjustment element includes a plurality of coils extending about the portion of the fluid passage.

### Embodiment 11

The system according to any prior embodiment, further including a thermocouple arranged in the body for monitoring a temperature of the fluid passage.

### Embodiment 12

The system according to any prior embodiment, further including an inner ring arranged about a portion of the fluid passage, the temperature adjustment element extending about at least a portion of the inner ring.

### Embodiment 13

The system according to any prior embodiment, wherein the thermocouple is arranged in the inner ring.

### Embodiment 14

The system according to any prior embodiment, wherein the sensor includes at least one sensor element extending into the fluid passage.

### Embodiment 15

The system according to any prior embodiment, wherein the fluid passage includes an inner surface extending from the inlet to the outlet, the inner surface being provided with a coating resistant to a material of concern.

### Embodiment 16

The system according to any prior embodiment, wherein the coating comprises an anti-scaling coating.

### Embodiment 17

The system according to any prior embodiment, wherein the zone of interest comprises a resource bearing formation.

### Embodiment 18

The system according to any prior embodiment, wherein the zone of interest comprises a fluid treatment system.

### Embodiment 19

The system according to any prior embodiment, wherein the fluid treatment system comprises a heat exchanger.

### Embodiment 20

The system according to any prior embodiment, wherein the sensor includes at least one sensor element, the temperature adjustment system being in thermal communication with the at least one sensor element.

The use of the terms “a” and “an” and “the” and similar referents in the context of describing the invention (especially in the context of the following claims) are to be construed to cover both the singular and the plural, unless otherwise indicated herein or clearly contradicted by context. Further, it should further be noted that the terms “first,” “second,” and the like herein do not denote any order, quantity, or importance, but rather are used to distinguish one element from another. The modifier “about” used in connection with a quantity is inclusive of the stated value and has the meaning dictated by the context (e.g., it includes the degree of error associated with measurement of the particular quantity).

The teachings of the present disclosure may be used in a variety of well operations. These operations may involve using one or more treatment agents to treat a formation, the fluids resident in a formation, a wellbore, and/or equipment in the wellbore, such as production tubing. The treatment agents may be in the form of liquids, gases, solids, semi-solids, and mixtures thereof. Illustrative treatment agents include, but are not limited to, fracturing fluids, acids, steam, water, brine, anti-corrosion agents, cement, permeability modifiers, drilling muds, emulsifiers, demulsifiers, tracers, flow improvers etc. Illustrative well operations include, but are not limited to, hydraulic fracturing, stimulation, tracer injection, cleaning, acidizing, steam injection, water flooding, cementing, water disposal, etc.

While the invention has been described with reference to an exemplary embodiment or embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the claims. Also, in the drawings and the description, there have been disclosed exemplary embodiments of the invention and, although specific terms may have been employed, they are unless otherwise stated used in a generic and descriptive sense only and not for purposes of limitation, the scope of the invention therefore not being so limited.

What is claimed is:

1. A sensor system comprising:
a body including an inlet, an outlet, and a fluid passage extending between the inlet and the outlet;
a sensor arranged at the fluid passage spaced from the outlet; and
a temperature adjustment element arranged in thermal communication with at least one of a portion of the fluid passage and the sensor.

2. The sensor system according to claim 1, wherein the temperature adjustment element includes a plurality of coils extending about the portion of the fluid passage.

3. The sensor system according to claim 1, further comprising: a thermocouple arranged in the body for monitoring a temperature of the fluid passage.

4. The sensor system according to claim 3, further comprising: an inner ring arranged about a portion of the fluid passage, the temperature adjustment element extending about at least a portion of the inner ring.

5. The sensor system according to claim 4, wherein the thermocouple is arranged in the inner ring.

6. The sensor system according to claim 1, wherein the sensor includes at least one sensor element extending into the fluid passage.

7. The sensor system according to claim 1, wherein the fluid passage includes an inner surface extending from the inlet to the outlet, the inner surface being provided with a coating resistant to a material of concern.

8. The sensor system according to claim 7, wherein the coating comprises an anti-scaling coating.

9. A system comprising:
a fluid source;
a passage extending from the fluid source, the passage including a zone of interest; and
a sensor system mounted to the passage remote from the zone of interest, the sensor system comprising:
a body including an inlet, an outlet, and a fluid passage extending between the inlet and the outlet;
a sensor arranged at the fluid passage spaced from the outlet; and
a temperature adjustment element arranged in thermal communication with at least one of a portion of the fluid passage and the sensor.

10. The system according to claim 9, wherein the temperature adjustment element includes a plurality of coils extending about the portion of the fluid passage.

11. The system according to claim 9, further comprising: a thermocouple arranged in the body for monitoring a temperature of the fluid passage.

12. The system according to claim 11, further comprising: an inner ring arranged about a portion of the fluid passage, the temperature adjustment element extending about at least a portion of the inner ring.

13. The system according to claim 12, wherein the thermocouple is arranged in the inner ring.

14. The system according to claim 9, wherein the sensor includes at least one sensor element extending into the fluid passage.

15. The system according to claim 9, wherein the fluid passage includes an inner surface extending from the inlet to the outlet, the inner surface being provided with a coating resistant to a material of concern.

16. The system according to claim 15, wherein the coating comprises an anti-scaling coating.

17. The system according to claim 9, wherein the zone of interest comprises a resource bearing formation.

18. The system according to claim 9, wherein the zone of interest comprises a fluid treatment system.

19. The system according to claim 18, wherein the fluid treatment system comprises a heat exchanger.

20. The system according to claim 9, wherein the sensor includes at least one sensor element, the temperature adjustment element being in thermal communication with the at least one sensor element.
